(19) United States
(12) Patent Application Publication
(10) Pub. No.: US 2024/0207229 A1
(43) Pub. Date: Jun. 27, 2024

(54) COCRYSTALLINE FORMS OF A BRUTON'S TYROSINE KINASE INHIBITOR

(71) Applicant: Loxo Oncology, Inc., Indianapolis, IN (US)

(72) Inventors: David Andrew COATES, New Palestine, IN (US); Lori Raquel HILDEN, Indianapolis, IN (US)

(21) Appl. No.: 18/555,467

(22) PCT Filed: May 11, 2022
(86) PCT No.: PCT/US2022/028663

§ 371 Date: Oct. 13, 2023

Related U.S. Application Data
(60) Provisional application No. 63/188,747, filed on May 14, 2021.

(51) Int. Cl. A61K 31/415; A61K 47/32; A61K 47/38; C07C 55/14; C07C 61/06; C07D 231/38

(52) U.S. Cl. CPC A61K 31/415; A61K 47/32; A61K 47/38; C07C 55/14; C07C 61/06; C07D 231/38; C07C 2601/08

(57) ABSTRACT

Provided herein are cocrystalline forms including BTK-I useful in the treatment and prevention of diseases which can be treated with a BTK inhibitor, including BTK-associated diseases and disorders, characterizations, and methods of making these cocrystalline forms.

The present disclosure relates to novel cocrystalline forms of a Bruton's Tyrosine Kinase (BTK) inhibitor, to pharmaceutical compositions comprising the cocrystalline forms, to methods of using the cocrystalline forms to treat conditions treatable by the inhibition of BTK, such as B-cell malignancy, B-cell lymphoma, marginal zone lymphoma (MZL), diffuse large B-cell lymphoma (DLBCL), chronic lymphocytic leukemia (CLL), small lymphocytic lymphoma (SLL), non-Hodgkin lymphoma, Burkitt lymphoma, mantle cell lymphoma (MCL), follicular lymphoma (FL), hairy cell leukemia, B-cell non-Hodgkin lymphoma, B-cell prolymphocytic leukemia, Waldenstrom's macroglobulinemia (WM), multiple myeloma (MM), arthritis, in particular rheumatoid arthritis (RA), and multiple sclerosis (MS) and to processes useful in the synthesis of the cocrystalline forms.

BTK is a molecular target useful for treatment across numerous B-cell leukemias and lymphomas including, for example, indolent and aggressive mature B cell non-Hodgkin lymphomas, CLL, SLL, WM, MCL, FL, DLBCL, B-cell prolymphocytic leukemia, hairy cell leukemia, and MZL. It has also been reported that B cells play a prominent role in the development of chronic graft versus host disease (cGVHD), a life-threatening complication of allogeneic stem cell transplantation, prompting studies of B cell-targeted therapies for the prevention and treatment of cGVHID.

BTK inhibitors are known in the art, for example, in WO 2013/010136, U.S. Pat. No. 9,090,621, WO 2015/127310, WO 2015/095099 and US 2014/221333.

Furthermore, in addition to cancer, it has been reported that certain BTK inhibitors are being studied in clinical trials for RA and/or MS, for example WO 2021/202825 and WO 2020/016850. The compound, (S)-5-amino-3-(4-((5-fluoro-2-methoxybenzamido)methyl)phenyl)-1-(1,1,1-trifluoropropane-2-yl)-1H-pyrazole-4-carboxamide (hereinafter referred to as “BTK-I”), and pharmaceutically acceptable salts thereof are disclosed in WO 2017/103611.

Further, a spray dried dispersion (SDD) formulation of BTK-I is disclosed in WO 2020/028258.

Novel forms of BTK-I are desired which provide solid state stability and chemical stability for the preparation and manufacture of pharmaceutical formulations. The pharmaceutical composition may include one or more polymers, for example polyvinylpyrrolidone vinyl acetate (PVP-VA), hydroxypropylmethylcellulose (HPMC) or hydroxypropylmethylcellulose acetate succinate (HPMCAS) such as HPMCAS-L, HPMCAS-M, or HPMCAS-H. The pharmaceutical composition may also include a pharmaceutically acceptable carrier, diluent, or excipient.

It is an aim of certain embodiments of the present disclosure to provide cocrystalline forms that are stable and have low hygroscopicity. It is an aim of certain embodiments of the present disclosure to utilize cocrystalline forms which require less operational process steps than current formulations. Cocrystalline forms may result in fewer operational process steps which includes the benefits of simplification of the supply chain and fewer number of unit operations. Cocrystalline forms undergo less material transfers from different sites, such as transferring once to incorporate the cocrystal into a formulation, such as tablets, capsules, and suspensions. It is generally recognized that SDD formulations start with manufacturing the Active Pharmaceutical Ingredient (API) at a first site, transfer to a second site for incorporation of the API in the SDD, and then transfer to a third site for incorporation of the SDD with API into a formulation, such as tablets, capsules, and suspensions. It is also an aim of certain embodiments of the present disclosure to utilize cocrystalline forms which require less solvent than current formulations leading to less environmental impact.

Accordingly, described herein are cocrystalline forms of BTK-I and pharmaceutical compositions thereof. Certain embodiments of the present disclosure satisfy some or all of the above aims.

In an aspect, disclosed herein is a cocrystalline form of BTK-I and a coformer selected from the group consisting of adipic acid and camphoric acid. In an embodiment of this aspect, disclosed herein is a cocrystalline form comprising BTK-I and adipic acid.

In another embodiment of the aspect, disclosed herein is the cocrystalline form wherein the ratio of (S)-5-amino-3-(4-((5-fluoro-2-methoxybenzamido)methyl)phenyl)-1-(1,1,1-trifluoropropane-2-yl)-1H-pyrazole-4-carboxamide to adipic acid is about 2:1 (herein referred to as “BTK-I and hemi-adipic acid cocrystalline form”).

In another embodiment of the aspect, disclosed herein is the BTK-I and hemi-adipic acid cocrystalline form characterized by having an x-ray powder diffraction (XRPD) pattern using CuKα radiation comprising a peak at 18.7° and one or more peaks at 8.1°, 10.4°, 11.7°, 12.1°, 14.2°, 15.1°, 17.0°, 17.3°, 18.1°, 19.2°, 19.9°, 20.4°, 20.9°, 21.6°, 22.1°, 23.7°, 24.3°, 24.8°, 25.5°, 26.1°, 27.2°, 27.4°, 28.3°, or 29.8° with a tolerance for the diffraction angles of ±0.2 degrees. In another embodiment of the aspect, disclosed herein is the BTK-I and hemi-adipic acid cocrystalline form characterized by an XRPD pattern using CuKα radiation having a diffraction peak at diffraction angle 2-theta of 18.7° in combination with one or more of the peaks selected from the group consisting of 10.4°, 14.2°, 15.1°, 17.0°, and 21.6°; with a tolerance for the diffraction angles of ±0.2 degrees. In another embodiment of the aspect, disclosed herein is the BTK-I and hemi-adipic acid cocrystalline form characterized by an XRPD pattern using CuKα radiation having a diffraction peak at diffraction angle 2-theta of 18.7° in combination with one or more of the peaks selected from the group consisting of 14.2°, 17.0°, and 21.6°; with a tolerance for the diffraction angles of ±0.2 degrees. In another embodiment of the aspect, disclosed herein is the BTK-I and hemi-adipic acid cocrystalline form characterized by an XRPD pattern using CuKα radiation having a diffraction peak at diffraction angle 2-theta of 18.7° in combination with one or more of the peaks selected from the group consisting of 17.0° and 21.6°; with a tolerance for the diffraction angles of ±0.2 degrees. In another embodiment of the aspect, disclosed herein is the cocrystalline form wherein the diffraction peak at diffraction angle 2-theta is 18.7°. In another embodiment of the aspect, disclosed herein is the cocrystalline form wherein the one or more peaks at diffraction angle 2-theta is selected from the group consisting of 17.0° and 21.6°.

In another embodiment of the aspect, disclosed herein is the BTK-I and hemi-adipic acid cocrystalline form characterized by a ¹³C solid state NMR spectrum of the BTK-I and hemi-adipic acid cocrystalline form comprises peaks referenced to the high field resonance of adamantane (δ=29.5 ppm) at: 174.6, 167.6, 166.1, 157.5, 155.5, 152.7, 150.9, 150.3, 141.1, 140.4, 130.1, 129.3, 127.7, 126.6, 123.6, 120.7, 120.2, 118.5, 116.5, 114.1, 112.8, 91.2, 63.6, 58.6, 56.8, 52.2, 44.0, 34.5, 33.2, 25.1, 24.7, 13.8, 13.2 ppm, with a tolerance of ±0.2 ppm. In another embodiment of the aspect, disclosed herein is the cocrystalline form characterized by ¹³C solid state NMR spectrum which comprises peaks referenced to the high field resonance of adamantane at 174.6, 91.2, 44.0, 13.8, and 13.2 ppm with a tolerance of ±0.2 ppm. In another embodiment of the aspect, disclosed herein is the BTK-I and hemi-adipic acid cocrystalline form which can be represented by the structures such as:

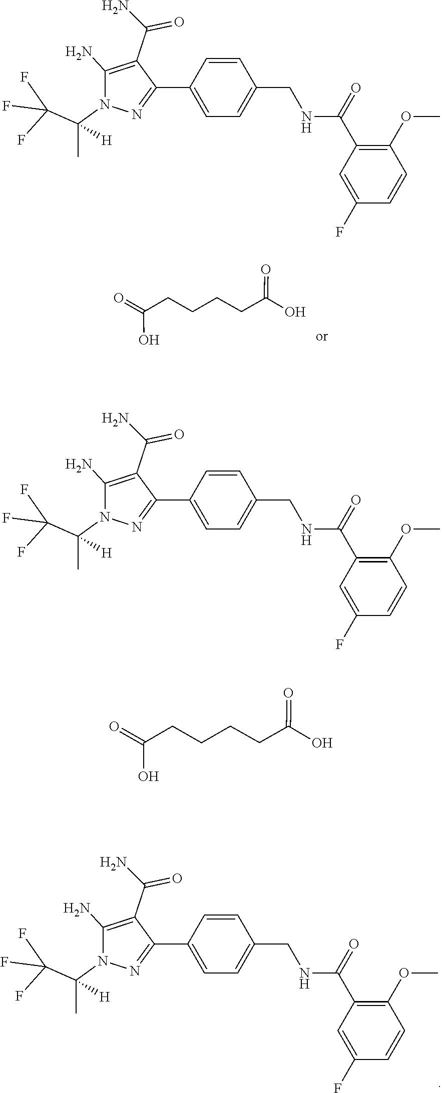 [SMILES: COc1ccc(F)cc1C(=O)NCc1ccc(-c2nn([C@@H](C)C(F)(F)F)c(N)c2C(N)=O)cc1 ; O=C(O)CCCCC(=O)O ; COc1ccc(F)cc1C(=O)NCc1ccc(-c2nn([C@@H](C)C(F)(F)F)c(N)c2C(N)=O)cc1 ; O=C(O)CCCCC(=O)O ; COc1ccc(F)cc1C(=O)NCc1ccc(-c2nn([C@@H](C)C(F)(F)F)c(N)c2C(N)=O)cc1]

In an embodiment of this aspect, disclosed herein is a cocrystalline form BTK-I and camphoric acid. In another embodiment of this aspect, disclosed herein is a cocrystalline form comprising (S)-5-amino-3-(4-((5-fluoro-2-methoxybenzamido)methyl)phenyl)-1-(1,1,1-trifluoropropane-2-yl)-1H-pyrazole-4-carboxamide and camphoric acid (herein referred to as “BTK-I and camphoric acid cocrystalline form”). In another embodiment of this aspect, disclosed herein is the BTK-I and camphoric acid cocrystalline form wherein the ratio of (S)-5-amino-3-(4-((5-fluoro-2-methoxybenzamido)methyl)phenyl)-1-(1,1,1-trifluoropropane-2-yl)-1H-pyrazole-4-carboxamide to camphoric acid is about 1:1 (herein referred to as “BTK-I and mono-camphoric acid cocrystalline form”). In another embodiment of this aspect, disclosed herein is the BTK-I and mono-camphoric acid cocrystalline form characterized by having an XRPD pattern using CuKα radiation comprising a peak at 17.7° and one or more peaks at 7.2°, 8.3°, 10.2°, 11.7°, 11.9°, 12.6°, 13.4°, 13.8°, 14.5°, 15.6°, 15.8°, 16.7°, 19.0°, 20.4°, 21.1°, 23.6°, 25.5°, 26.10, or 27.2°, with a tolerance of ±0.2 ppm. In another embodiment of this aspect, disclosed herein is the BTK-I and mono-camphoric acid cocrystalline form characterized by an XRPD pattern using CuKα radiation having a diffraction peak at diffraction angle 2-theta of 17.7° in combination with one or more of the peaks selected from the group consisting of 7.2°, 8.3°, 12.6°, 14.5°, and 16.7°; with a tolerance for the diffraction angles of ±0.2 degrees. In another embodiment of this aspect, disclosed herein is the BTK-I and mono-camphoric acid cocrystalline form characterized by an XRPD pattern using CuKα radiation having a diffraction peak at diffraction angle 2-theta of 17.7° in combination with one or more of the peaks selected from the group consisting of 7.2°, 14.5°, and 16.7°; with a tolerance for the diffraction angles of ±0.2 degrees. In another embodiment of this aspect, disclosed herein is the BTK-I and mono-camphoric acid cocrystalline form characterized by an XRPD pattern using CuKα radiation having a diffraction peak at diffraction angle 2-theta of 17.7° in combination with one or more of the peaks selected from the group consisting of 7.2° and 14.5°; with a tolerance for the diffraction angles of ±0.2 degrees. In another embodiment of this aspect, disclosed herein is the cocrystalline form wherein the diffraction peak at diffraction angle 2-theta is 17.7°. In another embodiment of this aspect, disclosed herein is the cocrystalline form wherein the one or more peaks at diffraction angle 2-theta is selected from the group consisting of 7.2° and 14.5°. In another embodiment of this aspect, disclosed herein is the BTK-I and mono-camphoric acid cocrystalline form which can be represented by a structure such as:

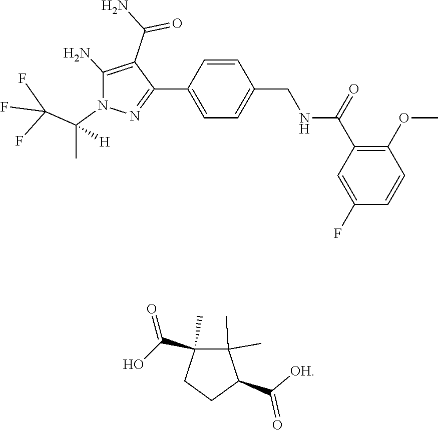 [SMILES: COc1ccc(F)cc1C(=O)NCc1ccc(-c2nn([C@@H](C)C(F)(F)F)c(N)c2C(N)=O)cc1 ; CC1(C)[C@@H](C(=O)O)CC[C@@]1(C)C(=O)O]

In another aspect, disclosed herein is a pharmaceutical composition comprising the cocrystalline form and further comprising one or more polymers. In an embodiment of this aspect, disclosed herein the polymer is PVP-VA, hydroxypropylmethylcellulose (HPMC), or HPMCAS such as HPMCAS-L, HPMCAS-M, or HPMCAS-H.

In another embodiment of this aspect, disclosed herein the pharmaceutical composition comprising a cocrystalline form may comprise about 90 weight parts cocrystalline form and about 10 weight parts polymer, about 80 weight parts cocrystalline form and about 20 weight parts polymer, about 70 weight parts cocrystalline form and about 30 weight parts polymer, or about 50 weight parts cocrystalline form and about 50 weight parts polymer. In another embodiment of this aspect, disclosed herein the pharmaceutical composition comprises about 80 weight parts cocrystalline form and about 20 weight parts polymer.

In another embodiment of this aspect, disclosed herein the pharmaceutical composition further comprises a pharmaceutically acceptable carrier, diluent, or excipient. In another embodiment of this aspect, disclosed herein is a pharmaceutical composition and a pharmaceutically acceptable carrier, diluent or excipient. In another embodiment of this aspect, disclosed herein is the pharmaceutical composition wherein the composition contains less than about 20% by wt. of other cocrystalline forms of BTK-I and a different coformer. In another embodiment of this aspect, disclosed herein is the pharmaceutical composition wherein the composition contains less than about 10% by wt. of other cocrystalline forms of BTK-I and a different coformer. In another embodiment of this aspect, disclosed herein is the pharmaceutical composition wherein the composition contains less than about 5% by wt. of other cocrystalline forms of BTK-I and a different coformer.

In another aspect disclosed herein is a method of treating cancer in a patient in need thereof comprising administering an effective amount of the cocrystal or pharmaceutical composition thereof of the present invention. In an embodiment of this aspect, disclosed herein is a method of treating a BTK associated cancer in a patient in need thereof comprising administering BTK-I and hemi-adipic acid cocrystalline form and the BTK-I. In another embodiment of this aspect, disclosed herein is a method of treating a BTK associated cancer in a patient in need thereof comprising administering BTK-I and mono-camphoric acid cocrystalline form. In another embodiment of this aspect, disclosed herein the BTK associated cancer is selected from the group consisting of B-cell malignancy, B-cell lymphoma, MZL, DLBCL, CLL, SLL, non-Hodgkin lymphoma, Burkitt lymphoma, MCL, FL, hairy cell leukemia, B-cell non-Hodgkin lymphoma, WM, B-cell prolymphocytic leukemia and MM. In another embodiment of this aspect, disclosed herein the BTK associated cancer is MCL. In another embodiment of this aspect, disclosed herein the BTK associated cancer is CLL. In another embodiment of this aspect, disclosed herein the BTK associated cancer is SLL. In another embodiment of this aspect, disclosed herein the BTK associated cancer is FL. In another embodiment of this aspect, disclosed herein the BTK associated cancer is MZL. In another embodiment of this aspect, disclosed herein the MZL is splenic, nodal, or extranodal. In another embodiment of this aspect, disclosed herein the BTK associated cancer is DLBCL. In another embodiment of this aspect, disclosed herein the BTK associated cancer is: B-cell non-Hodgkin lymphoma, MCL, CLL, or SLL. In another embodiment of this aspect, disclosed herein the BTK associated cancer is selected from the group consisting of: MCL, CLL, SLL, WM, FL, and MZL.

In another aspect, disclosed herein is a method of inhibiting Bruton's tyrosine kinase in a patient in need thereof, comprising administering to the patient an effective amount of the cocrystal or pharmaceutical composition thereof of the present invention.

In another aspect, disclosed herein is a method of treating MS in a patient in need thereof comprising administering an effective amount of a cocrystal or pharmaceutical composition thereof of the present invention.

In another aspect disclosed herein is a method of treating arthritis, more particularly RA, in a patient in need thereof comprising administering an effective amount of the cocrystal or pharmaceutical composition thereof of the present invention.

In another aspect, disclosed herein is the cocrystal or the pharmaceutical composition thereof of the present invention for use in therapy. In an embodiment of this aspect, disclosed herein is the cocrystal or the pharmaceutical composition thereof of the present invention for use in the treatment of cancer. In another embodiment of this aspect, disclosed herein is BTK-I and hemi-adipic acid cocrystalline form for use in the treatment of a BTK associated cancer. In another embodiment of this aspect, disclosed herein is BTK-I and mono-camphoric acid cocrystalline form for use in the treatment of a BTK associated cancer. In another embodiment of this aspect, disclosed herein the BTK associated cancer is selected from the group consisting of B-cell malignancy, B-cell lymphoma, MZL, DLBCL, CLL, SLL, non-Hodgkin lymphoma, Burkitt lymphoma, MCL, FL, hairy cell leukemia, B-cell non-Hodgkin lymphoma, WM, B-cell prolymphocytic leukemia and MM. In another embodiment of this aspect, disclosed herein the BTK associated cancer is MCL. In another embodiment of this aspect, disclosed herein the BTK associated cancer is CLL. In another embodiment of this aspect, disclosed herein the BTK associated cancer is SLL. In another embodiment of this aspect, disclosed herein the BTK associated cancer is FL. In another embodiment of this aspect, disclosed herein the BTK associated cancer is MZL. In another embodiment of this aspect, disclosed herein the MZL is splenic, nodal, or extranodal. In another embodiment of this aspect, disclosed herein the BTK associated cancer is DLBCL. In another embodiment of this aspect, disclosed herein the BTK associated cancer is B-cell non-Hodgkin lymphoma, MCL, CLL, or SLL. In another embodiment of this aspect, disclosed herein the BTK associated cancer is selected from the group consisting of: MCL, CLL, SLL, WM, FL, and MZL.

In another aspect disclosed herein is the cocrystal or pharmaceutical composition thereof of the present invention for use in inhibiting Bruton's tyrosine kinase.

In another aspect disclosed herein is the cocrystal or the pharmaceutical composition thereof for use in the treatment of MS.

In another aspect disclosed herein is the cocrystal or pharmaceutical composition thereof for use in the treatment of arthritis, in particular RA.

In another aspect, disclosed herein is the use of the cocrystal or the pharmaceutical composition thereof of the present invention in the manufacture of a medicament for the treatment of cancer. In an embodiment of this aspect, disclosed herein is the use of BTK-I and hemi-adipic acid cocrystalline form in the manufacture of a medicament for the treatment of a BTK associated cancer. In another embodiment of this aspect, disclosed herein is the use of BTK-I and mono-camphoric acid cocrystalline form in the manufacture of a medicament for the treatment of a BTK associated cancer. In another embodiment of this aspect, disclosed herein the BTK associated cancer is selected from the group consisting of B-cell malignancy, B-cell lymphoma, MZL, DLBCL, CLL, SLL, non-Hodgkin lymphoma, Burkitt lymphoma, MCL, FL, hairy cell leukemia, B-cell non-Hodgkin lymphoma, WM, B-cell prolymphocytic leukemia and MM. In another embodiment of this aspect, disclosed herein the BTK associated cancer is MCL. In another embodiment of this aspect, disclosed herein the BTK associated cancer is CLL. In another embodiment of this aspect, disclosed herein the BTK associated cancer is SLL. In another embodiment of this aspect, disclosed herein the BTK associated cancer is FL. In another embodiment of this aspect, disclosed herein the BTK associated cancer is MZL. In another embodiment of this aspect, disclosed herein the MZL is splenic, nodal, or extranodal. In another embodiment of this aspect, disclosed herein the BTK associated cancer is DLBCL. In another embodiment of this aspect, disclosed herein the BTK associated cancer is: B-cell non-Hodgkin lymphoma, MCL, CLL, or SLL. In another embodiment of this aspect, disclosed herein the BTK associated cancer is selected from the group consisting of: MCL, CLL, SLL, WM, FL, and MZL.

In another aspect, disclosed herein is the use of the cocrystal or pharmaceutical composition thereof of the present invention in the manufacture of a medicament for inhibiting Bruton's tyrosine kinase.

In another aspect, disclosed herein is use of the cocrystal or pharmaceutical composition thereof in the manufacture of a medicament for the treatment of MS.

In another aspect, disclosed herein is the use of the cocrystal or pharmaceutical composition thereof in the manufacture of a medicament for the treatment of arthritis, in particular RA.

In another aspect, disclosed herein is a process for the preparation of BTK-I and hemi-adipic acid cocrystalline form comprising the steps of suspending and partially dissolving adipic acid in a BTK-I and hemi-adipic acid cocrystalline form solvent, adding BTK-I and stirring with heat, and isolating the BTK-I and hemi-adipic acid cocrystalline form. In an embodiment of this aspect, disclosed herein is the process wherein the step of isolating is by filtration under vacuum followed by drying under a nitrogen stream. In another embodiment of this aspect, disclosed herein is the process wherein the heat is at about 55° C. In another embodiment of this aspect, disclosed herein is the process wherein the step of stirring is at about 500 rpm. In another embodiment of this aspect, disclosed herein is the process wherein the step of stirring with heat occurs for about one hour. In another embodiment of this aspect, disclosed herein is the process wherein the BTK-I and hemi-adipic acid cocrystalline form solvent is selected from the group consisting of ethyl acetate, cyclopentyl methyl ether, isopropyl alcohol, and acetonitrile. In another embodiment of this aspect, disclosed herein is the process wherein the BTK-I and hemi-adipic acid cocrystalline form solvent is ethyl acetate.

In another aspect, disclosed herein is a process for the preparation of the BTK-I and mono-camphoric acid cocrystalline form comprising the steps of suspending BTK-I in a BTK-I and mono-camphoric acid cocrystalline solvent saturated with camphoric acid, stirring with heat followed by stirring without heat; and isolating the BTK-I and mono-camphoric acid cocrystalline form. In an embodiment of this aspect, disclosed herein is the process wherein the step of isolating is by filtration under vacuum. In another embodiment of this aspect, disclosed herein is the process wherein the heat is at about 50° C. In another embodiment of this aspect, disclosed herein is the process wherein the step of stirring is at about 800 rpm. In another embodiment of this aspect, disclosed herein is the process wherein the step of stirring with heat occurs for about two hours. In another embodiment of this aspect, disclosed herein is the process wherein the step of stirring without heat occurs for more than two hours. In another embodiment of this aspect, disclosed herein is the process wherein the BTK-I and mono-camphoric acid cocrystalline solvent is cyclopentyl methyl ether.

In another aspect, disclosed herein is a cocrystalline form of BTK-I obtainable by any of the processes of the present invention.

### BRIEF DESCRIPTION OF THE DRAWINGS

FIG. 1 is an x-ray powder diffraction overlay of BTK-I crystalline free form (bottom), BTK-I and hemi-adipic acid cocrystalline form (middle), and BTK-I and camphoric acid cocrystalline form (top).

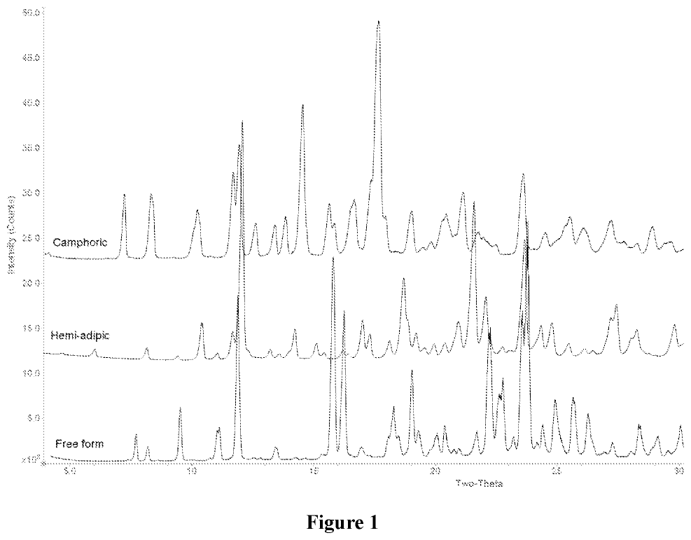

### DEFINITIONS

Unless defined otherwise, all technical and scientific terms used herein have the meaning commonly understood by a person skilled in the art to which this disclosure belongs. As used herein, the following terms have the meanings ascribed to them below, unless specified otherwise.

Adipic acid, also known as hexanedioic acid, has the structure illustrated in the formula below:

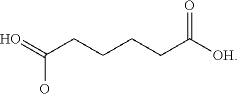

Camphoric acid, also known as rel-(1R,3S)-1,2,2-trimethylcyclopentane-1,3-dicarboxylic acid or (1R,3S)-rel-1,2,2-trimethylcyclopentane-1,3-dicarboxylic acid, has the structure illustrated in the formula below:

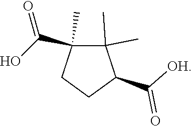

The compound, BTK-I, also known as 5-amino-3-[4-[[(5-fluoro-2-methoxy-benzoyl)amino]methyl]phenyl]-1-[(1S)-2,2,2-trifluoro-1-methyl-ethyl]pyrazole-4-carboxamide or (S)-5-amino-3-(4-((5-fluoro-2-methoxybenzamido)methyl)phenyl)-1-(1,1,1-trifluoropropan-2-yl)-1H-pyrazole-4-carboxamide, has the structure illustrated below:

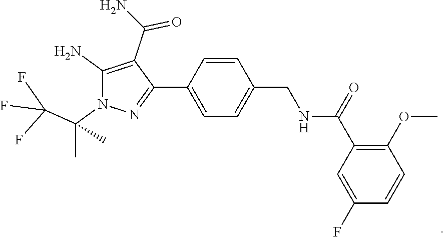

BTK-I may be prepared as described in U.S. Pat. No. 10,342,780.

The terms “treatment,” “treat,” “treating,” and the like, are meant to include slowing, stopping, or reversing the progression of a disorder. These terms also include alleviating, ameliorating, attenuating, eliminating, or reducing one or more symptoms of a disorder or condition, even if the disorder or condition is not actually eliminated and even if progression of the disorder or condition is not itself slowed, stopped, or reversed.

The term “effective amount” means an amount of a compound that is capable of providing a therapeutic benefit to the patient in need thereof. The effective amount in a particular patient may be affected by factors such as whether a compound or its salt will be administered; the patient's size, age, gender, and general health; the stage and/or the severity of the cancer, illness, or disease; the responsiveness of the individual patient to prior therapy; whether the patient has a reoccurrence of the disease, illness or cancer after a prior therapy; the mode of administration; the bioavailability characteristics of the preparation administered; the dose regimen selected; and the use of other concomitant medication.

The term “therapeutic benefit” means an improvement in survival, a reduction of the symptoms, a restoration of functional capacity or a lowering of the chances of developing a chronic condition. Measurements of such therapeutic benefits include increases in overall survival, progression-free survival, time to progression, disease-free survival, event-free survival, time to treatment failure, time to next treatment, duration of clinical benefit, duration of response, objective response rate, complete response, pathological complete response, disease control rate, clinical benefit rate, health-related quality of life and milestone survival. See A. Delgado and A. K. Guddati, Clinical Endpoints in Oncology—a Primer, Am J Cancer Res, 2021; 11(4): 1121-1131. As used herein, the term “patient” refers to a human.

To provide a more concise description, some of the quantitative expressions herein are recited as a range from about amount X to about amount Y. It is understood that when a range is recited, the range is not limited to the recited upper and lower bounds, but rather includes the full range from about amount X through about amount Y, or any range therein.

“Room temperature” or “RT” refers to the ambient temperature of a typical laboratory, which is typically around 25° C.

As used herein, the term “excipient” refers to any substance needed to formulate the composition to a desired form. For example, suitable excipients include but are not limited to, diluents or fillers, binders or granulating agents or adhesives, disintegrants, lubricants, antiadherants, glidants, dispersing or wetting agents, dissolution retardants or enhancers, adsorbents, buffers, chelating agents, preservatives, colors, flavors and sweeteners.

A “pharmaceutically acceptable carrier, diluent, or excipient” is a medium generally accepted in the art for the delivery of biologically active agents to humans. The term “pharmaceutically acceptable carrier” or “pharmaceutically acceptable excipient” includes any and all solvents, co-solvents, complexing agents, dispersion media, coatings, films, antibacterial and antifungal agents, isotonic and absorption delaying agents, and the like, which are not biologically or otherwise undesirable. The use of such media and agents for pharmaceutically active substances is well known in the art. Except insofar as any conventional media or agent is incompatible with the active ingredient, its use in the therapeutic formulations is contemplated. Supplementary active ingredients can also be incorporated into the formulations. In addition, various excipients, such as are commonly used in the art, can be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (2010); Goodman and Gilman's: The Pharmacological Basis of Therapeutics, 12th Ed., The McGraw-Hill Companies.

As used herein, the singular forms “a,” “an,” and “the” include plural referents unless the context clearly dictates otherwise.

As used herein, ranges and amounts can be expressed as “about” a particular value or range, in particular “about” means within 5% or 10% of the numerical number. About also includes the exact amount. Hence, “about 5 grams” means “about 5 grams” and also “5 grams.” It also is understood that ranges expressed herein include whole numbers within the ranges and fractions thereof. For example, a range of between 5 grams and 20 grams includes whole number values such as 5, 6, 7, 8, 9, 10, 11, 12, 13, 14, 15, 16, 17, 18, 19, 20, 21 and 22 grams, and fractions within the range including, but not limited to, 4.5, 4.75, 5.25, 6.5, 8.75, 11.95, and 21.95 grams.

As used herein, “optional” or “optionally” means that the subsequently described event or circumstance does or does not occur and that the description includes instances where said event or circumstance occurs and instances where it does not. For example, a reaction mixture that “optionally includes a catalyst” means that the reaction mixture contains a catalyst or it does not contain a catalyst.

As used herein, “relative intensity” means the percentage of any peak in relation to the highest peak in the relevant spectrum.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination.

All combinations of the embodiments pertaining to the aspects described herein are specifically embraced by the present disclosure just as if each and every combination was individually explicitly recited, to the extent that such combinations embrace possible aspects. In addition, all sub-combinations of the embodiments contained within the aspects described herein, as well as all sub-combinations of the embodiments contained within all other aspects described herein, are also specifically embraced by the present disclosure just as if each and every sub-combination of all embodiments are explicitly recited herein.

The following examples further illustrate the disclosure.

The XRPD patterns are obtained on the instruments described in Example 1 and Example 2. For each, the dry powder is packed on a quartz sample holder and a smooth surface is obtained using a glass slide. The crystal form diffraction patterns are collected at ambient temperature and relative humidity. Crystal peak positions are determined in MDI-Jade after whole pattern shifting based on an internal NIST 675 standard with peaks at 8.853 and 26.774 20°.

For any given crystal form, it is well known in the crystallography art that, for the relative intensities of the diffraction peaks may vary due to preferred orientation resulting from factors such as crystal morphology and habit. Where the effects of preferred orientation are present, peak intensities are altered, but the characteristic peak positions of the forms are unchanged. See, e.g. The United States Pharmacopeia #23, National Formulary #18, pages 1843-1844, 1995. Furthermore, it is also well known in the crystallography art that for any given crystal form the angular peak positions may vary slightly. For example, peak positions can shift due to a variation in the temperature at which a sample is analyzed, sample displacement, or the presence or absence of an internal standard. In the present case, a peak position variability of ±0.2 2θ° is presumed to take into account these potential variations without hindering the unequivocal identification of the indicated crystal form. Confirmation of a crystal form may be made based on any unique combination of distinguishing peaks.

Solid state NMR is obtained on Bruker Avance III HD with a Bruker Ultrashield 400WB Plus magnet operating at a frequency of 100.6 MHz. The probe employed is a Bruker MAS 4 BL CP BB DVT N-P/H. Acquisitional parameters are as follows: 31104 scans, 34 ms acquisition time, 4.6 s interpulse delay, 10 kHz MAS frequency, 1.5 ms contact time, and a SPINAL64 decoupling scheme. The data are externally referenced to adamantane at 29.5 ppm±0.2 ppm.

### Example 1

### (S)-5-Amino-3-(4-((5-fluoro-2-methoxybenzamido)methyl)phenyl)-1-(1,1,1-trifluoropropane-2-yl)-1H-pyrazole-4-carboxamide (“BTK-I”), Hemi-Adipic Acid Cocrystalline form (“BTK-I and hemi-adipic acid cocrystalline form”)

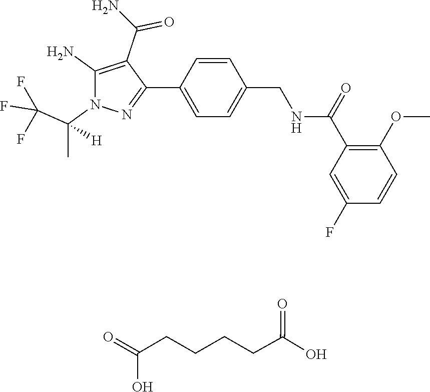 [SMILES: COc1ccc(F)cc1C(=O)NCc1ccc(-c2nn([C@@H](C)C(F)(F)F)c(N)c2C(N)=O)cc1 ; O=C(O)CCCCC(=O)O]

Adipic acid (2.42 g, 16.56 mmol) in ethyl acetate (60 mL) is suspended and partially dissolved and the mixture is heated to 55° C. (S)-5-Amino-3-(4-((5-fluoro-2-methoxybenzamido)methyl)phenyl)-1-(1,1,1-trifluoropropane-2-yl)-1H-pyrazole-4-carboxamide (15.08 g, 31.45 mmol) is added and the mixture is stirred at 500 rpm at 55° C. The solids never fully dissolve, but over the next few minutes the slurry changes from a light tan to a bright white. The reaction is stirred for 1 hour and the heat source is removed. The white solid is isolated on a nylon filter under vacuum and dried under a nitrogen stream for 15 minutes. The solid is dried in a vacuum oven at 65° C. for 48 hours to give the title compound (16.3 g, 94.3%). ¹³C Solid state NMR (101 MHz) for the BTK-I and hemi-adipic acid cocrystalline form includes peaks at δ 174.6, 167.6, 166.8, 166.1, 157.5, 155.5, 152.7, 150.9, 150.3, 141.1, 140.4, 132.1, 130.1, 129.3, 127.7, 126.6, 123.6, 120.7, 120.2, 118.5, 116.5, 114.1, 112.8, 91.2, 63.6, 58.6, 56.8, 52.2, 44.0, 34.5, 33.2, 25.1, 24.7, 13.8, 13.2.

### XRPD of BTK-I and Hemi-Adipic Acid Cocrystalline Form

The XRPD pattern of BTK-I and hemi-adipic acid cocrystalline form solids is obtained on a Bruker D4 Endeavor X-ray powder diffractometer, equipped with a CuKα (1.5418 Å) source and a Vantec detector, operating at 35 kV and 50 mA. The BTK-I and hemi-adipic acid cocrystalline form sample is scanned between 4 and 40 2θ°, with a step size of 0.008 2θ° and a scan rate of 0.5 seconds/step, and using 1.0 mm divergence, 6.6 mm fixed anti-scatter, and 11.3 mm detector slits.

### XRPD of BTK-I and Hemi-Adipic Acid Cocrystalline Form

A prepared sample of BTK-I and hemi-adipic acid cocrystalline form is characterized by an XRPD pattern using CuKα radiation as having diffraction peaks (2-theta values) as described in Table 1 below, and in particular having a peak at 18.7 in combination with one or more of the peaks selected from the group consisting of 14.2, 17.0, and 21.6; with a tolerance for the diffraction angles of ±0.2 degrees.

TABLE 1
XRPD peaks of Example 1

| | | Relative Intensity |
|---|---|---|
| Peak | Angle (°2-Theta) +/− 0.2° | (% of most intense peak) |
| 1 | 8.1 | 4.8 |
| 2 | 10.4 | 15.7 |
| 3 | 11.7 | 11.5 |
| 4 | 12.1 | 100.0 |
| 5 | 14.2 | 12.5 |
| 6 | 15.1 | 6.4 |
| 7 | 17.0 | 15.9 |
| 8 | 17.3 | 10.0 |
| 9 | 18.1 | 7.1 |
| 10 | 18.7 | 34.2 |
| 11 | 19.2 | 10.4 |
| 12 | 19.9 | 5.8 |
| 13 | 20.4 | 6.3 |
| 14 | 20.9 | 15.2 |
| 15 | 21.6 | 66.1 |
| 16 | 22.1 | 25.9 |
| 17 | 23.7 | 49.7 |
| 18 | 24.3 | 13.7 |
| 19 | 24.8 | 15.0 |
| 20 | 25.5 | 6.1 |
| 21 | 26.1 | 3.8 |
| 22 | 27.2 | 15.5 |
| 23 | 27.4 | 21.0 |
| 24 | 28.3 | 10.5 |
| 25 | 29.8 | 13.2 |

### Alternate Example 1

### BTK-I and Hemi-Adipic Acid Cocrystalline Form

Alternatively, BTK-I and hemi-adipic acid cocrystalline form can be made using solvents other than ethyl acetate, for example isopropyl alcohol as the solvent may be done as the following: (S)-5-Amino-3-(4-((5-fluoro-2-methoxybenzamido)methyl)phenyl)-1-(1,1,1-trifluoropropane-2-yl)-1H-pyrazole-4-carboxamide (1.75 g, 3.65 mmol) and adipic acid (0.266 g, 1.82 mmol) are suspended in isopropyl alcohol (20 mL). The slurry is heated to 80° C. All solids are dissolved. The solution is cooled to 65° C. and is then seeded with 1 wt % cocrystal. The mixture is cooled to 55° C. over 8 hours using a linear cooling ramp. Stirring is maintained at 200 rpm. Solids are isolated onto a Whatman® #1 filter using vacuum filtration to afford the title compound (1.37 g, 68.3%)

### Example 2

### (S)-5-Amino-3-(4-((5-fluoro-2-methoxybenzamido)methyl)phenyl)-1-(1,1,1-trifluoropropane-2-yl)-1H-pyrazole-4-carboxamide, (1R,3S)-1,2,2-trimethylcyclopentane-1,3-dicarboxylic acid (“camphoric acid”) (“BTK-I and mono-camphoric acid cocrystalline form”)

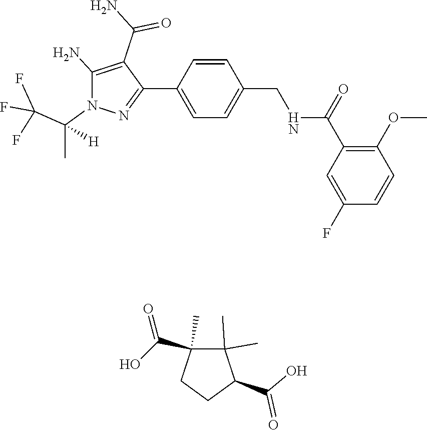 [SMILES: COc1ccc(F)cc1C(=O)NCc1ccc(-c2nn([C@@H](C)C(F)(F)F)c(N)c2C(N)=O)cc1 ; CC1(C)[C@@H](C(=O)O)CC[C@@]1(C)C(=O)O]

(S)-5-Amino-3-(4-((5-fluoro-2-methoxybenzamido)methyl)phenyl)-1-(1,1,1-trifluoropropane-2-yl)-1H-pyrazole-4-carboxamide (466 mg, 0.97 mmol) is suspended in cyclopentyl methyl ether (20 mL) saturated with camphoric acid and stirring at 800 rpm at 50° C. The reaction becomes a slurry of white solid (initially slightly tan). The mixture is stirred as a slurry for two hours at 50° C. The heat source is removed and the mixture is stirred at room temperature overnight. The white solid is isolated by filtration under vacuum to give the title compound (564 mg, 87.4% yield).

### XRPD of BTK-I and Mono-Camphoric Acid Cocrystalline Form

The XRPD pattern of BTK-I and mono-camphoric acid cocrystalline form solids is obtained on a Bruker D8 Endeavor X-ray powder diffractometer, equipped with a CuKα (1.5418 Å) source and a Linxeye detector, operating at 40 kV and 40 mA. The BTK-I and mono-camphoric acid cocrystalline form sample is scanned between 4 and 42 2θ°, with a step size of 0.009 2θ° and a scan rate of 0.5 seconds/step, and using 0.3° primary slit opening, and 3.9° position sensitive detector (PSD) opening.

### XRPD of BTK-I, (1R,3S)-1,2,2-trimethylcyclopentane-1,3-dicarboxylic acid

A prepared sample of BTK-I, (1R,3S)-1,2,2-trimethylcyclopentane-1,3-dicarboxylic acid is characterized by an XRPD pattern using CuKα radiation as having diffraction peaks (2-theta values) as described in Table 2, and in particular having a peak at 17.7 in combination with one or more of the peaks selected from the group consisting of 7.2, 14.5, and 16.7; with a tolerance for the diffraction angles of ±0.2 degrees.

TABLE 2
XRPD peaks of Example 2

| | | Relative Intensity |
|---|---|---|
| Peak | Angle (°2-Theta) +/− 0.2° | (% of most intense peak) |
| 1 | 7.2 | 28.2% |
| 2 | 8.3 | 28.2% |
| 3 | 10.2 | 21.2% |
| 4 | 11.7 | 37.2% |
| 5 | 11.9 | 49.4% |
| 6 | 12.6 | 14.0% |
| 7 | 13.4 | 12.6% |
| 8 | 13.8 | 15.6% |
| 9 | 14.5 | 65.1% |
| 10 | 15.6 | 22.5% |
| 11 | 15.8 | 13.7% |
| 12 | 16.7 | 22.0% |
| 13 | 17.7 | 100.0% |
| 14 | 19.0 | 18.0% |
| 15 | 20.4 | 14.9% |
| 16 | 21.1 | 25.4% |
| 17 | 23.6 | 35.5% |
| 18 | 25.5 | 11.6% |
| 19 | 26.1 | 10.8% |
| 20 | 27.2 | 13.6% |

What is claimed is:

1-34. (canceled)

35. A crystalline form of (S)-5-amino-3-(4-((5-fluoro-2-methoxybenzamido)methyl)phenyl)-1-(1,1,1-trifluoropropane-2-yl)-1H-pyrazole-4-carboxamide comprising the following formula:

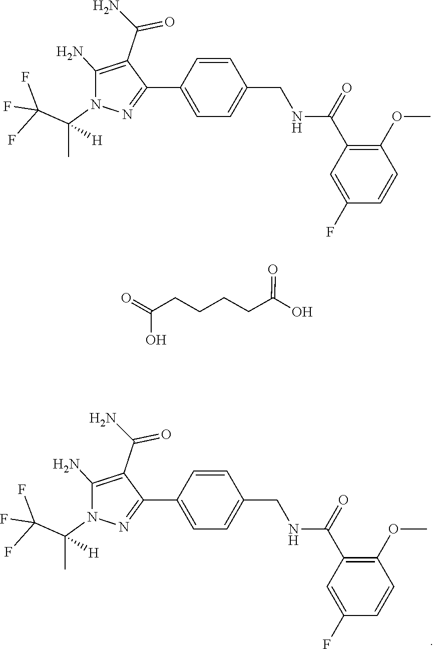 [SMILES: COc1ccc(F)cc1C(=O)NCc1ccc(-c2nn([C@@H](C)C(F)(F)F)c(N)c2C(N)=O)cc1 ; O=C(O)CCCCC(=O)O ; COc1ccc(F)cc1C(=O)NCc1ccc(-c2nn([C@@H](C)C(F)(F)F)c(N)c2C(N)=O)cc1]

36. The crystalline form of claim 35, having an X-ray powder diffraction (XRPD) pattern comprising an XRPD peak at 2-theta angle of 18.7°±0.2°.

37. The crystalline form of claim 36, having an X-ray powder diffraction (XRPD) pattern further comprising one or more XRPD peaks at 2-theta angles of 8.1°±0.2°, 10.4°±0.2°, 11.7°±0.2°, 12.1°±0.2°, 14.2°±0.2°, 15.1°±0.2°, 17.0°±0.2°, 17.3°±0.2°, 18.1°±0.2°, 19.2°±0.2°, 19.9°±0.2°, 20.4°±0.2°, 20.9°±0.2°, 21.6°±0.2°, 22.1°±0.2°, 23.7°±0.2°, 24.3°±0.2°, 24.8°±0.2°, 25.5°±0.2°, 26.1°±0.2°, 27.2°±0.2°, 27.4°±0.2°, 28.3°±0.2°, or 29.8°±0.2.

38. The crystalline form of claim 36, having an X-ray powder diffraction (XRPD) pattern comprising XRPD peaks at 2-theta angle of 18.7°±0.2°, 17.0°±0.2°, and 21.6°±0.2°.

39. The crystalline form of claim 36, having an X-ray powder diffraction (XRPD) pattern comprising XRPD peaks at 2-theta angle of 18.7°±0.2°, 14.2°±0.2°, 17.0°±0.2°, and 21.6°±0.2°.

40. A pharmaceutical composition comprising the crystalline form of claim 35 and a pharmaceutically acceptable carrier, diluent, and/or excipient.

41. The pharmaceutical composition of claim 40, comprising one or more polymers selected from the group consisting of polyvinylpyrrolidone vinyl acetate (PVP-VA), hydroxypropylmethylcellulose (HPMC) or hydroxypropylmethylcellulose acetate succinate (HPMCAS).

42. The pharmaceutical composition of claim 41, wherein the HPMCAS is selected from the group consisting of HPMCAS-L, HPMCAS-M, and HPMCAS-H.

43. A method of treating a Bruton's Tyrosine Kinase (BTK)-associated cancer in a patient in need thereof, comprising administering to the patient an effective amount of the crystalline form of claim 35.

44. The method of claim 43, wherein the BTK-associated cancer is selected from B-cell malignancy, B-cell lymphoma, marginal zone lymphoma, diffuse large B-cell lymphoma, chronic lymphocytic leukemia, small lymphocytic lymphoma, non-Hodgkin lymphoma, Burkitt lymphoma, mantle cell lymphoma, follicular lymphoma, hairy cell leukemia, B-cell non-Hodgkin lymphoma, B-cell prolymphocytic leukemia, Waldenstrom's macroglobulinemia, and multiple myeloma.

45. The method of claim 44, wherein the BTK-associated cancer is chronic lymphocytic leukemia.

46. The method of claim 44, wherein the BTK-associated cancer is small lymphocytic lymphoma.

47. The method of claim 44, wherein the BTK-associated cancer is mantle cell lymphoma.

48. A method of treating multiple sclerosis in a patient in need thereof, comprising administering to the patient an effective amount of the crystalline form of claim 35.

49. A method of treating arthritis in a patient in need thereof, comprising administering to the patient an effective amount of the crystalline form of claim 35.

50. The method of claim 49, wherein the arthritis is rheumatoid arthritis.

51. A process for preparing the crystalline form of claim 35, comprising:
mixing (S)-5-amino-3-(4-((5-fluoro-2-methoxybenzamido)methyl)phenyl)-1-(1,1,1-trifluoropropane-2-yl)-1H-pyrazole-4-carboxamide and adipic acid in a solvent;
and heating the mixture to about 55° C.

52. The process of claim 51, wherein the solvent is selected from the group consisting of ethyl acetate, cyclopentyl methyl ether, isopropyl alcohol, and acetonitrile.

53. The process of claim 51, wherein the mixture is stirred at about 500 rpm.

54. The process of claim 52, further comprising isolating the crystalline form of claim 35.

55. The process of claim 53, further comprising isolating the crystalline form of claim 35.

56. A process for preparing the crystalline form of claim 35, comprising:
mixing (S)-5-amino-3-(4-((5-fluoro-2-methoxybenzamido)methyl)phenyl)-1-(1,1,1-trifluoropropane-2-yl)-1H-pyrazole-4-carboxamide and adipic acid in a solvent;
and heating the mixture to about 80° C.

57. The process of claim 56, wherein the mixture is cooled to about 65° C. and seeded with 1 wt % of the crystalline form of claim 1.

58. The process of claim 57, further comprising isolating the crystalline form of claim 1.

59. A crystalline form of (S)-5-amino-3-(4-((5-fluoro-2-methoxybenzamido)methyl)phenyl)-1-(1,1,1-trifluoropropane-2-yl)-1H-pyrazole-4-carboxamide comprising the following formula:

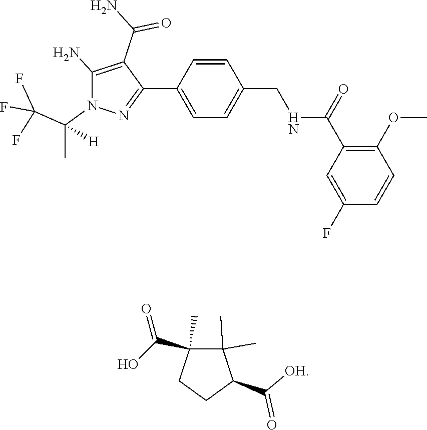 [SMILES: COc1ccc(F)cc1C(=O)NCc1ccc(-c2nn([C@@H](C)C(F)(F)F)c(N)c2C(N)=O)cc1 ; CC1(C)[C@@H](C(=O)O)CC[C@@]1(C)C(=O)O]

60. The crystalline form of claim 59, having an X-ray powder diffraction (XRPD) pattern comprising an XRPD peak at 2-theta angle of 17.7°±0.2°.

61. The crystalline form of claim 60, having an X-ray powder diffraction (XRPD) pattern further comprising one or more XRPD peaks at 2-theta angles of 7.2°±0.2°, 8.3°±0.2°, 10.2°±0.2°, 11.7°±0.2°, 11.9°±0.2°, 12.6°±0.2°, 13.4°±0.2°, 13.8°±0.2°, 14.5°±0.2°, 15.6°±0.2°, 15.8°±0.2°, 16.7°±0.2°, 19.0°±0.2°, 20.4°±0.2°, 21.1°±0.2°, 23.6°±0.2°, 25.5°±0.2°, 26.1°±0.2°, or 27.2°±0.2.

62. The crystalline form of claim 60, having an X-ray powder diffraction (XRPD) pattern comprising XRPD peaks at 2-theta angle of 7.2°±0.2°, 14.5°±0.2°, 16.7°±0.2°, and 17.7°±0.2°.

63. The crystalline form of claim 60, having an X-ray powder diffraction (XRPD) pattern comprising XRPD peaks at 2-theta angle of 7.2°±0.2°, 14.5±0.2°, and 17.7°±0.2°.

64. A pharmaceutical composition comprising the crystalline form of claim 59 and a pharmaceutically acceptable carrier, diluent, and/or excipient.

65. The pharmaceutical composition of claim 64, comprising one or more polymers selected from the group consisting of polyvinylpyrrolidone vinyl acetate (PVP-VA), hydroxypropylmethylcellulose (HPMC) or hydroxypropylmethylcellulose acetate succinate (HPMCAS).

66. The pharmaceutical composition of claim 65, wherein the HPMCAS is selected from the group consisting of HPMCAS-L, HPMCAS-M, and HPMCAS-H.

67. A method of treating a Bruton's Tyrosine Kinase (BTK)-associated cancer in a patient in need thereof, comprising administering to the patient an effective amount of the crystalline form of claim 59.

68. The method of claim 67, wherein the BTK-associated cancer is selected from B-cell malignancy, B-cell lymphoma, marginal zone lymphoma, diffuse large B-cell lymphoma, chronic lymphocytic leukemia, small lymphocytic lymphoma, non-Hodgkin lymphoma, Burkitt lymphoma, mantle cell lymphoma, follicular lymphoma, hairy cell leukemia, B-cell non-Hodgkin lymphoma, B-cell prolymphocytic leukemia, Waldenstrom's macroglobulinemia, and multiple myeloma.

69. The method of claim 68, wherein the BTK-associated cancer is chronic lymphocytic leukemia.

70. The method of claim 68, wherein the BTK-associated cancer is small lymphocytic lymphoma.

71. The method of claim 68, wherein the BTK-associated cancer is mantle cell lymphoma.

72. A method of treating multiple sclerosis in a patient in need thereof, comprising administering to the patient an effective amount of the crystalline form of claim 59.

73. A method of treating arthritis in a patient in need thereof, comprising administering to the patient an effective amount of the crystalline form of claim 59.

74. The method of claim 73, wherein the arthritis is rheumatoid arthritis.

75. A process for preparing the crystalline form of claim 59, comprising:
mixing (S)-5-amino-3-(4-((5-fluoro-2-methoxybenzamido)methyl)phenyl)-1-(1,1,1-trifluoropropane-2-yl)-1H-pyrazole-4-carboxamide and camphoric acid in a solvent;
and heating the mixture to about 50° C.

76. The process of claim 75, wherein the solvent is selected from the group consisting of ethyl acetate, cyclopentyl methyl ether, isopropyl alcohol, and acetonitrile.

77. The process of claim 75, wherein the mixture is stirred at about 800 rpm.

78. The process of claim 76, further comprising isolating the crystalline form of claim 59.

79. The process of claim 77, further comprising isolating the crystalline form of claim 59.
